(19) United States
(12) Patent Application Publication
(10) Pub. No.: US 2020/0304777 A1
(43) Pub. Date: Sep. 24, 2020

(54) DISPLAY DEVICE, MEDICAL OBSERVATION SYSTEM, DISPLAY METHOD, AND COMPUTER READABLE RECORDING MEDIUM

(71) Applicant: Sony Olympus Medical Solutions Inc., Tokyo (JP)

(72) Inventor: Takaaki YAMADA, Kanagawa (JP)

(73) Assignee: Sony Olympus Medical Solutions Inc., Tokyo (JP)

(21) Appl. No.: 16/783,194

(22) Filed: Feb. 6, 2020

(30) Foreign Application Priority Data
Mar. 22, 2019 (JP) 2019-055739

(51) Int. Cl. H04N 13/359; H04N 13/332; H04N 13/122; A61B 90/00; A61B 90/50

(52) U.S. Cl. CPC H04N 13/359; H04N 13/332; H04N 13/122; A61B 2090/367; A61B 90/37; A61B 90/50; A61B 90/361

(57) ABSTRACT

A display device for observing a three-dimensional image or a two-dimensional image through stereoscopic glasses includes: a display panel configured to display a three-dimensional image based on a three-dimensional image signal or a two-dimensional image based on a two-dimensional image signal; circuitry configured to determine whether or not an input image signal is the three-dimensional image signal; change a brightness of an image to be displayed on the display panel to a brightness suitable for the two-dimensional image when it is determined that the input image signal is not the three-dimensional image signal; and control the display panel to display an image based on the image signal with the changed brightness.

This application claims priority from Japanese Application No. 2019-055739, filed on Mar. 22, 2019, the contents of which are incorporated by reference herein in its entirety.

### BACKGROUND

The present disclosure relates to a display device, a medical observation system, a display method, and a computer readable recording medium.

As a medical observation system for observing a minute part of a brain, heart, or the like of a patient that is an object to be observed when performing an operation on the minute part, a video-type microscope system including an imaging unit which magnifies and captures an image of a minute part such as an operated site has been known (for example, see JP 2016-59499 A). In this microscope system, two optical systems each receive a formed subject image and photoelectrically convert the subject image to generate two imaging signals having mutual parallax, and a three-dimensional image (hereinafter, simply referred to as the 3D image) based on the two imaging signals is displayed on a display device, and an operator observes the 3D image while wearing stereoscopic glasses (hereinafter, simply referred to as the “3D glasses”).

### SUMMARY

In JP 2016-59499 A described above, the 3D image is displayed in a state in which a brightness of a display monitor is adjusted so that the operator can appropriately observe the image while wearing the 3D glasses. In other words, observation of a two-dimensional image (hereinafter, simply referred to as a 2D image) based on any one of the two imaging signals is not considered. When switching from a 3D image to a 2D image, the 2D image is displayed with the brightness for the 3D image. As a result, when the operator observes the 2D image with the naked eye, the 2D image is too bright and cannot be properly observed.

According to one aspect of the present disclosure, there is provided a display device for observing a three-dimensional image or a two-dimensional image through stereoscopic glasses, the display device including: a display panel configured to display a three-dimensional image based on a three-dimensional image signal or a two-dimensional image based on a two-dimensional image signal; circuitry configured to determine whether or not an input image signal is the three-dimensional image signal; change a brightness of an image to be displayed on the display panel to a brightness suitable for the two-dimensional image when it is determined that the input image signal is not the three-dimensional image signal; and control the display panel to display an image based on the image signal with the changed brightness.

### BRIEF DESCRIPTION OF THE DRAWINGS

FIG. 1 is a view illustrating a configuration of a medical observation system according to an embodiment;

FIG. 2 is an enlarged perspective view illustrating a configuration of a microscope unit of an observation apparatus according to an embodiment and the vicinity thereof;

FIG. 3 is a diagram schematically illustrating a situation of an operation performed using the medical observation system according to an embodiment;

FIG. 4 is a block diagram illustrating a functional configuration of a display device according to an embodiment; and

FIG. 5 is a flowchart illustrating an overview of processing performed by the display device according to an.

### DETAILED DESCRIPTION

Hereinafter, embodiments for carrying out the present disclosure (hereinafter, referred to as embodiments) will be described with reference to the accompanying drawings. Note that the drawings are merely schematic, and portions for which the relationships between dimensions and the proportions are different among drawings may be included in the drawings.

Configuration of Medical Observation System

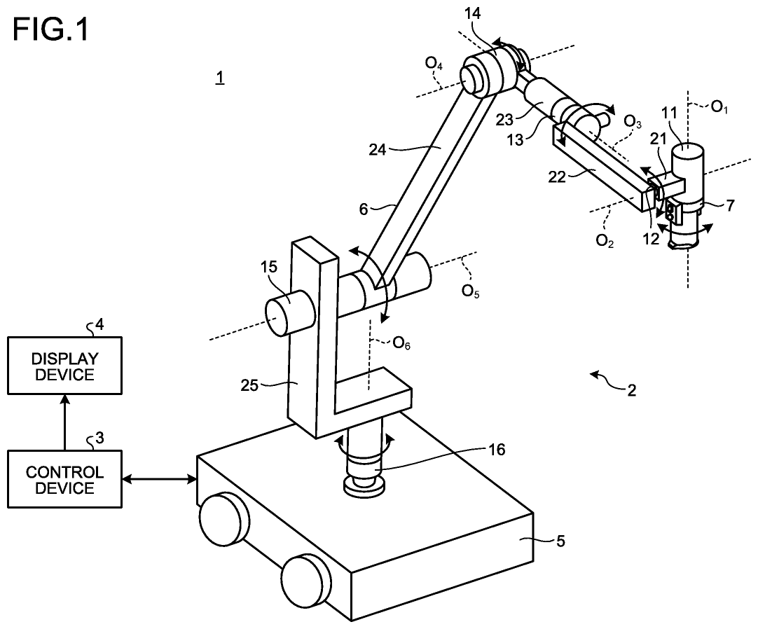

FIG. 1 is a view illustrating a configuration of a medical observation system according to an embodiment. A medical observation system 1 illustrated in FIG. 1 includes a medical observation apparatus 2 (hereinafter, referred to as the “observation apparatus 2”) having a function as a microscope that magnifies and captures an image of a minute structure of an object to be observed, a control device 3 which comprehensively controls operation of the medical observation system 1, and a display device 4 which displays the image captured by the observation apparatus 2.

The observation apparatus 2 includes a base unit 5 that is movable on a floor surface, a support unit 6 supported by the base unit 5, and a columnar microscope unit 7 provided at a distal end of the support unit 6 and magnifying and capturing an image of a minute part of the object to be observed.

The support unit 6 includes a first joint unit 11, a first arm unit 21, a second joint unit 12, a second arm unit 22, a third joint unit 13, a third arm unit 23, a fourth joint unit 14, a fourth arm unit 24, a fifth joint unit 15, a fifth arm unit 25, and a sixth joint unit 16.

The support unit 6 includes four sets each including two arm units and a joint unit that rotatably connect one (distal end side) of the two arm units to the other one (proximal end side). Specifically, these four sets are (the first arm unit 21, the second joint unit 12, and the second arm unit 22), (the second arm unit 22, the third joint unit 13, and the third arm unit 23), (the third arm unit 23, the fourth joint unit 14, and the fourth arm unit 24), and (the fourth arm unit 24, the fifth joint unit 15, and the fifth arm unit 25).

The first joint unit 11 has a distal end rotatably holding the microscope unit 7 and a proximal end held by the first arm unit 21 in a state of being fixed to a distal end portion of the first arm unit 21. The first joint unit 11 has a circular cylindrical shape and holds the microscope unit 7 so as to be rotatable around a first axis O₁ which is a central axis in a height direction. The first arm unit 21 has a shape extending from a side surface of the first joint unit 11 in a direction orthogonal to the first axis O₁. A more specific configuration of the first joint unit 11 will be described later.

The second joint unit 12 has a distal end rotatably holding the first arm unit 21 and a proximal end held by the second arm unit 22 in a state of being fixed to a distal end portion of the second arm unit 22. The second joint unit 12 has a circular cylindrical shape and holds the first arm unit 21 so as to be rotatable around a second axis O₂ which is a central axis in the height direction and is orthogonal to the first axis O₁. The second arm unit 22 has a substantial “L”-letter shape and an end portion of a vertical line portion of the “L”-letter shape is connected to the second joint unit 12.

The third joint unit 13 has a distal end rotatably holding a horizontal line portion of the “L”-letter shape of the second arm unit 22, and a proximal end held by the third arm unit 23 in a state of being fixed to a distal end portion of the third arm unit 23. The third joint unit 13 has a circular cylindrical shape and holds the second arm unit 22 so as to be rotatable around a third axis O₃ which is a central axis in the height direction, is orthogonal to the second axis O₂, and is parallel to a direction in which the second arm unit 22 extends. The distal end of the third arm unit 23 has a circular cylindrical shape and a hole that penetrates in a direction orthogonal to a height direction of the circular cylindrical distal end is formed at a proximal end of the third arm unit 23. The third joint unit 13 is rotatably held by the fourth joint unit 14 through this hole.

The fourth joint unit 14 has a distal end rotatably holding the third arm unit 23 and a proximal end held by the fourth arm unit 24 in a state of being fixed to the fourth arm unit 24. The fourth joint unit 14 has a circular cylindrical shape and holds the third arm unit 23 so as to be rotatable around a fourth axis O₄ which is a central axis in the height direction and is orthogonal to the third axis O₃.

The fifth joint unit 15 has a distal end rotatably holding the fourth arm unit 24 and a proximal end fixedly attached to the fifth arm unit 25. The fifth joint unit 15 has a circular cylindrical shape and holds the fourth arm unit 24 so as to be rotatable around a fifth axis O₅ which is a central axis in the height direction and is parallel to the fourth axis O₄. The fifth arm unit 25 includes a portion with an “L”-letter shape and a rod-shaped portion extending downward from a horizontal line portion of the “L”-letter shape. The proximal end of the fifth joint unit 15 is attached to an end portion of a vertical line portion of the “L”-letter shape of the fifth arm unit 25.

The sixth joint unit 16 has a distal end rotatably holding the fifth arm unit 25 and a proximal end fixedly attached to an upper surface of the base unit 5. The sixth joint unit 16 has a circular cylindrical shape and holds the fifth arm unit 25 so as to be rotatable around a sixth axis O₆ which is a central axis in the height direction and is orthogonal to the fifth axis O₅. A proximal end portion of the rod-shaped portion of the fifth arm unit 25 is attached to the distal end of the sixth joint unit 16.

The support unit 6 having the above-described configuration implements movement with a total of 6 degrees of freedom, that is, 3 degrees of freedom of translation and 3 degrees of freedom of rotation, for the microscope unit 7.

The first joint unit 11 to the sixth joint unit 16 have electromagnetic brakes that prohibit rotation of the microscope unit 7 and the first arm unit 21 to the fifth arm unit 25, respectively. Each electromagnetic brake is released in a state in which an arm operation switch 73 (described later) provided in the microscope unit 7 is pressed, and allows rotation of the microscope unit 7 and the first arm unit 21 to the fifth arm unit 25. Note that an air brake may be applied instead of the electromagnetic brake.

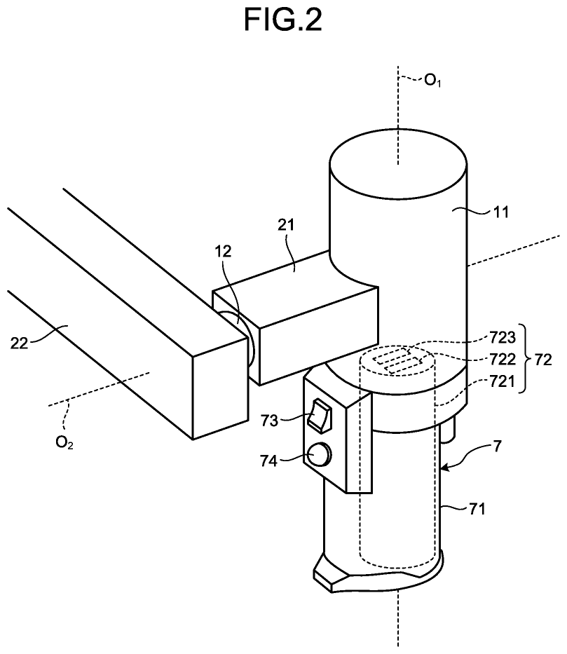

Here, a configuration of the microscope unit 7 of the observation apparatus 2 and the vicinity thereof will be described. FIG. 2 is an enlarged perspective view illustrating a configuration of the microscope unit 7 of the observation apparatus 2 and the vicinity thereof.

The microscope unit 7 includes a cylindrical unit 71 having a circular cylindrical shape, an imaging unit 72 that is provided in a hollow portion of the cylindrical unit 71 and magnifies and captures an image of the object to be observed, the arm operation switch 73 that receives an operation input that releases the electromagnetic brakes of the first joint unit 11 to the sixth joint unit 16 to allow the rotation of each joint unit, a cross lever 74 that can change a magnification and a focal length to the object to be observed in the imaging unit 72, an upper cover 75 formed around an upper portion of the imaging unit 72 and fitted in the first joint unit 11, and a shaft unit 76 having a hollow circular cylindrical shape and extending from the upper cover 75 along the first axis O₁.

The cylindrical unit 71 has a circular cylindrical shape with a diameter smaller than that of the first joint unit 11, and a cover glass (not illustrated) for protecting the imaging unit 72 is provided on an opened surface of a lower end portion of the cylindrical unit 71. Note that the shape of the cylindrical unit 71 is not limited to the circular cylindrical shape, and may be, for example, a cylindrical shape of which a cross section orthogonal to the height direction has an ellipse shape or a polygonal shape.

The imaging unit 72 includes an optical system 721 which includes a plurality of lenses arranged so that an optical axis of each of the lenses coincides with the first axis O₁, and collects light from the object to be observed and forms an image, and two image sensors 722 and 723 each of which receives the light collected by the optical system 721 and photoelectrically converts the light to generate an imaging signal. Note that only a cylindrical casing that houses the plurality of lenses of the optical system 721 is described in FIG. 2.

The optical system 721 can change the magnification of the image of the object to be observed and the focal length to the object to be observed according to the operation of the cross lever 74.

The image sensors 722 and 723 each are implemented by a charge coupled device (CCD), a complementary metal oxide semiconductor (CMOS), or the like. The image sensors 722 and 723 generate two imaging signals having mutual parallax as imaging signals for generating a 3D image. These imaging signals are output from the image sensors 722 and 723 as digital signals, respectively.

The arm operation switch 73 is a push-button switch. While a user keeps the arm operation switch 73 pressed, the electromagnetic brakes of the first joint unit 11 to the sixth joint unit 16 are released. The arm operation switch 73 is provided on a side surface opposite to a side surface facing the user when operating the microscope unit 7, in other words, on the side surface that is a blind spot of the user when operating the microscope unit 7. The arm operation switch 73 is a part of an operation input unit that receives an operation input to the observation apparatus 2.

The cross lever 74 can be operated along a height direction of the cylindrical unit 71 and a circumferential direction orthogonal to the height direction. The cross lever 74 is provided on a side surface of the cylindrical unit 71 below the arm operation switch 73 along the height direction of the cylindrical unit 71. Similarly to the arm operation switch 73, the cross lever 74 is a part of the operation input unit that receives an operation input to the observation apparatus 2, as well.

When the cross lever 74 is operated from a position illustrated in FIG. 2 along the height direction of the cylindrical unit 71, the magnification is changed, and when the cross lever 74 is operated from the position illustrated in FIG. 2 along the circumferential direction of the cylindrical unit 71, the focal length to the object to be observed is changed. For example, when the cross lever 74 is moved upward along the height direction of the cylindrical unit 71, the magnification is increased, and when the cross lever 74 is moved downward along the height direction of the cylindrical unit 71, the magnification is decreased. Further, when the cross lever 74 is moved clockwise along the circumferential direction of the cylindrical unit 71, the focal length to the object to be observed is increased, and the cross lever 74 is moved counterclockwise along the circumferential direction of the cylindrical unit 71, the focal length to the object to be observed is decreased. Note that assignment of movement directions and operations of the cross lever 74 is not limited to that described above.

Next, referring back to FIG. 1, the configuration of the medical observation system 1 will be described.

The control device 3 receives the imaging signal output from the observation apparatus 2, and performs predetermined signal processing on the imaging signal to generate three-dimensional image data for display. The control device 3 is implemented by a processor including a memory and hardware such as a central processing unit (CPU), a read only memory (ROM), and a random access memory (RAM). Note that the control device 3 may be installed inside the base unit 5 and integrated with the observation apparatus 2.

The display device 4 receives, from the control device 3, a 3D image signal (three-dimensional image data) or a 2D image signal (two-dimensional image data) generated by the control device 3, and display a 3D image based on the 3D image signal, or a 2D image based on the 2D image signal. Such a display device 4 includes a display panel formed of liquid crystal or organic electro luminescence (EL). Note that a specific configuration of the display device 4 will be described later.

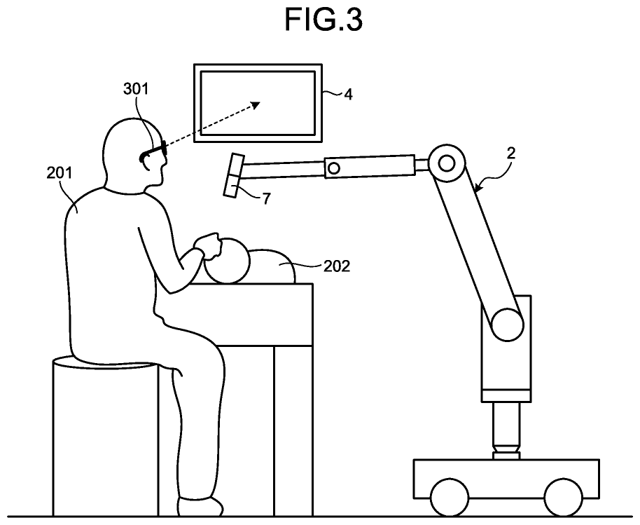

Next, an overview of an operation performed using the medical observation system 1 having the above-described configuration will be described. FIG. 3 is a diagram schematically illustrating a situation of an operation performed using the medical observation system 1. Specifically, FIG. 3 is a diagram schematically illustrating a situation where an operator 201 who is a user is performing an operation on the head of a patient 202 that is an object to be observed.

As illustrated in FIG. 3, the operator 201 grips the microscope unit 7, moves the microscope unit 7 to a desired position in a state of keeping the arm operation switch 73 of the microscope unit 7 pressed and determines an imaging visual field of the microscope unit 7, and then removes his/her finger from the arm operation switch 73, while wearing stereoscopic glasses 301 (hereinafter, simply referred to as “3D glasses 301”) for three-dimensional images and visually observing a 3D image displayed on the display device 4. Here, the 3D glasses are one of active shutter type (frame sequential type) glasses and passive type (circular polarization filter type) glasses, preferably, passive type glasses. In addition, when the display device 4 displays a 2D image, the operator 201 observes the 2D image with the 3D glasses 301 removed.

Thereby, in the first joint unit 11 to the sixth joint unit 16, the electromagnetic brakes are operated and the imaging visual field of the microscope unit 7 is fixed. Then, the operator 201 performs, for example, adjustment of a magnification and a focal length to the object to be observed. Since the display device 4 displays a three-dimensional image, the operator 201 can grasp an operated site three-dimensionally through the three-dimensional image.

In order for the operator 201 to easily grip the microscope unit 7 and in order to prevent blocking of a field of view when the operator 201 views the display device 4 or the operated site of the patient 202, it is more preferable that, for example, an outer diameter of the cylindrical unit 71 is approximately 40 to 70 mm, a distance between a focal point O of the microscope unit 7 and a lower end of the microscope unit 7 is approximately 150 to 600 mm, and a combined height of the microscope unit 7 and the first joint unit 11 is approximately 100 to 220 mm.

Specific Configuration of Display Device

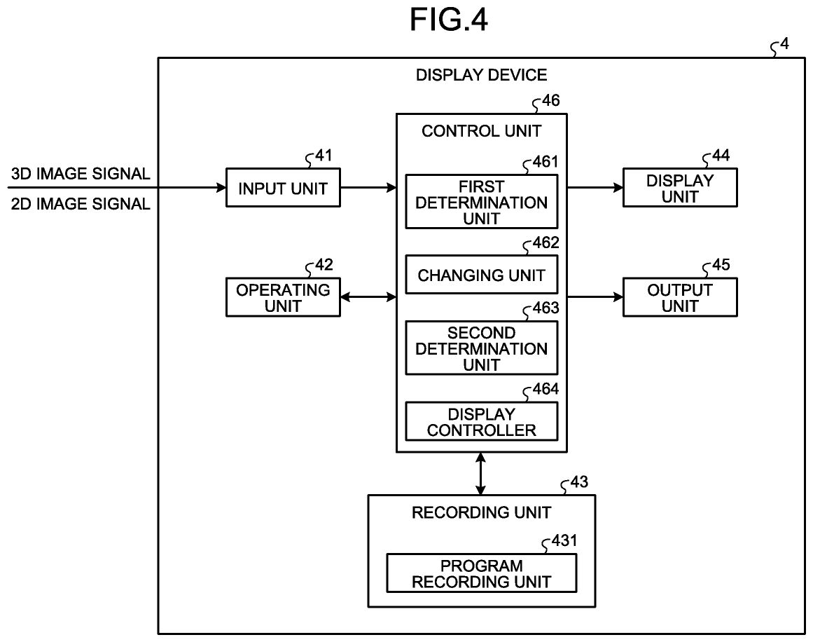

Next, a specific configuration of the display device 4 described in FIG. 1 will be described. FIG. 4 is a block diagram illustrating a functional configuration of the display device 4.

As illustrated in FIG. 4, the display device 4 includes an input unit 41, an operating unit 42, a recording unit 43, a display unit 44, an output unit 45, and a control unit 46.

The input unit 41 outputs an image signal input from the control device 3 to the control unit 46. Specifically, the input unit 41 receives, from the control device 3, an image signal of any one of 3D image data and 2D image data, or the like. The input unit 41 is implemented by, for example, an input and output interface.

The operating unit 42 receives an input for an operation of each component and outputs a signal corresponding to an operation indicated by the input to the control unit 46. The operating unit 42 is implemented by switches, buttons, a touch pad, and the like.

The recording unit 43 includes a program recording unit 431 which records various programs executed by the display device 4. The recording unit 43 is implemented by a volatile memory, a non-volatile memory, and the like.

The display unit 44 displays a 3D image based on the 3D image data input from the control unit 46 or a 2D image based on the 2D image data according to the control of the control unit 46. The display unit 44 is implemented by a liquid crystal display panel, an organic EL display panel, or the like.

The output unit 45 outputs sound based on sound data input from the control unit 46 according to the control of the control unit 46. The output unit 45 is implemented by a speaker or the like.

The control unit 46 comprehensively controls each unit of the display device 4. The control unit 46 is implemented by a processor including a memory and hardware such as a CPU, an application specific integrated circuit (ASIC), a field programmable gate array (FPGA), and a graphics processing unit (GPU). The control unit 46 includes a first determination unit 461, a changing unit 462, a second determination unit 463, and a display controller 464.

The first determination unit 461 determines whether or not an image signal input from the outside via the input unit 41 is 3D image data. Specifically, the first determination unit 461 determines whether or not the image signal input from the outside via the input unit 41 is 3D image data, according to the type of image signal. For example, the first determination unit 461 determines whether or not an image format of the image signal input via the input unit 41 is any one of a frame packing format, a multiview video coding (MVC) format, a side-by-side format, and a top-and-bottom format. When it is determined that the image format of the image signal is any one of these formats, the first determination unit 461 determines that the image signal input from the outside via the input unit 41 is 3D image data, and when it is determined that the image format of the image signal is not any one of these formats, the first determination unit 461 determines that the image signal input from the outside via the input unit 41 is 2D image data.

When the first determination unit 461 determines that the image signal input via the input unit 41 is not 3D image data, the changing unit 462 changes a brightness of an image to be displayed on the display unit 44 to a brightness suitable for 2D image based on 2D image data. Specifically, the changing unit 462 changes a brightness of the display unit 44 so that the image is displayed with a brightness twice that for a 3D image. In addition, when the first determination unit 461 determines that the image signal is 3D image data and the second determination unit 463 as described later determines that the brightness of the image to be displayed on the display unit 44 is equal to or lower than a predetermined threshold, the changing unit 462 changes the brightness of the image to be displayed on the display unit 44 to a brightness suitable for 3D image data. Specifically, the changing unit 462 changes the brightness of the display unit 44 so that the image is displayed with a brightness that is half that for a 2D image.

The second determination unit 463 determines whether or not the brightness of the image to be displayed on the display unit 44 is equal to or lower than the predetermined threshold. Specifically, the second determination unit 463 determines whether or not the brightness of the image to be displayed on the display unit 44 is equal to or lower than the brightness with which 3D image data are displayed.

The display controller 464 causes the display unit 44 to display an image based on an image signal with the brightness of the display unit 44 changed by the changing unit 462. Specifically, the display controller 464 causes the display unit 44 to display a 3D image or a 2D image with a brightness suitable for a 3D image based on 3D image data or a brightness suitable for a 2D image based on 2D image data.

Processing of Display Device

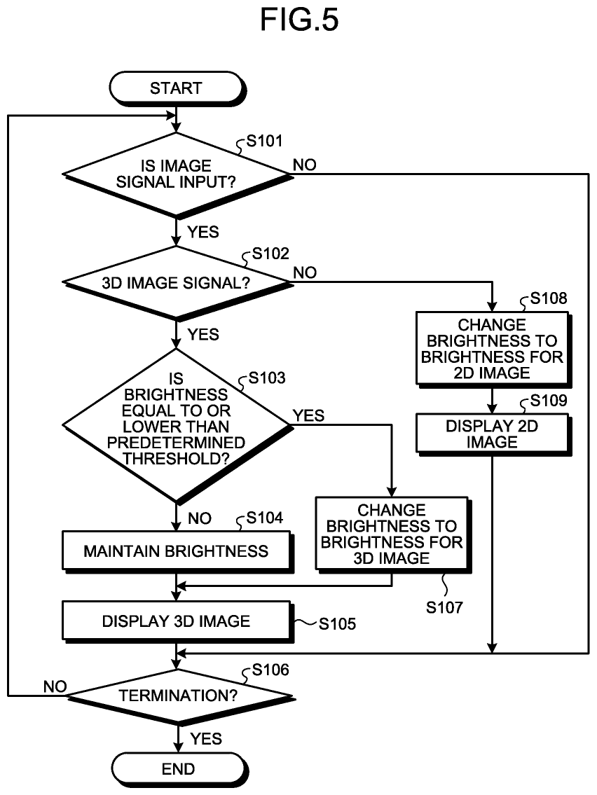

Next, processing performed by the display device 4 will be described. FIG. 5 is a flowchart illustrating an overview of processing performed by the display device 4.

As illustrated in FIG. 5, when an image signal is input from the outside via the input unit 41 (Step S101: Yes), the first determination unit 461 determines whether or not the image signal is a 3D image signal (Step S102). When the first determination unit 461 determines that the image signal is a 3D image signal (Step S102: Yes), the display device 4 proceeds to Step S103 described later. On the other hand, when the first determination unit 461 determines that the image signal is not a 3D image signal (Step S102: No), the display device 4 proceeds to Step S107 described later.

In Step S103, the second determination unit 463 determines whether or not a brightness of an image to be displayed on the display unit 44 is equal to or lower than a predetermined threshold. Specifically, the second determination unit 463 determines whether or not the brightness of the image to be displayed on the display unit 44 is equal to or lower than a reference brightness with which a 3D image signal is displayed. When the second determination unit 463 determines that the brightness of the image to be displayed on the display unit 44 is equal to or lower than the predetermined threshold (Step S103: Yes), the display device 4 proceeds to Step S104 described later. On the other hand, when the second determination unit 463 determines that the brightness of the image to be displayed on the display unit 44 is not equal to or lower than the predetermined threshold (Step S103: No), the display device 4 proceeds to Step S106 described later.

In Step S104, the changing unit 462 maintains the brightness of the image to be displayed on the display unit 44.

Next, the display controller 464 causes the display unit 44 to display a 3D image based on the 3D image signal with the brightness of the display unit 44 changed by the changing unit 462 (Step S105).

Then, when an instruction signal for instructing termination of observation is input from the operating unit 42 (Step S106: Yes), the display device 4 terminates the processing. On the other hand, when the instruction signal for instructing termination of observation is not input from the operating unit 42 (Step S106: No), the display device 4 returns to Step S101 described above.

In Step S107, the changing unit 462 changes the brightness of the image to be displayed on the display unit 44. Specifically, the changing unit 462 changes the brightness of the image to be displayed on the display unit 44 to a brightness suitable for a 3D image. For example, the changing unit 462 changes the brightness of the display unit 44 so that the image is displayed with a brightness that is half that for a 2D image. After Step S107, the display device 4 proceeds to Step S105.

In Step S108, the brightness of the image to be displayed on the display unit 44 is changed to a brightness suitable for a 2D image based on 2D image data. Specifically, the changing unit 462 changes a brightness of the display unit 44 so that the image is displayed with a brightness twice that for a 3D image.

Next, the display controller 464 causes the display unit 44 to display a 2D image based on a 2D image signal with the brightness of the display unit 44 changed by the changing unit 462 (Step S109). After Step S109, the display device 4 proceeds to Step S106.

In Step S101, when an image signal is not input from the outside via the input unit 41 (Step S101: No), the display device 4 proceeds to Step S106.

According to the embodiment described above, when the first determination unit 461 determines that an image signal input via the input unit 41 is not a 3D image signal, the changing unit 462 changes a brightness of an image to be displayed on the display unit 44 to a brightness suitable for a 2D image and the display controller 464 causes the display unit 44 to display the image based on the image signal with the brightness changed by the changing unit 462, such that it is possible to display an image with an appropriate brightness even when the type of image is switched.

Further, according to the embodiment, when the first determination unit 461 determines that an image signal is a 3D image and the second determination unit 463 determines that a brightness of an image to be displayed on the display unit 44 is equal to or lower than a predetermined threshold, the changing unit 462 changes the brightness of the image to be displayed on the display unit 44 to a brightness suitable for a 3D image, such that it is possible to prevent color from disappearing even in a case of switching from a 2D image to a 3D image, and it is possible to maintain a color space even when the type of image is switched. Further, since it is not necessary to change the brightness each time the type of image is switched, it is possible to prevent interference with the operation.

Moreover, according to an embodiment, since the 3D glasses are passive type glasses, inconvenience of the operator can be reduced.

Note that in the medical observation system according to the embodiment described above, it may be sufficient that the support unit 6 includes at least one set including two arm units and a joint unit that rotatably connects one of the two arm units to the other one.

Further, in the medical observation system according to an embodiment, the operation input unit provided in the cylindrical unit 71 is not limited to that described above. For example, an operating unit for changing a magnification and an operating unit for changing a focal length to the object to be observed may be provided separately.

Further, in the medical observation system according to an embodiment, the medical observation apparatus may be arranged so as to be suspended from a ceiling of a place where the medical observation apparatus is installed.

Moreover, variations can be conceived by appropriately combining a plurality of components of the medical observation system according to an embodiment. For example, some of all components of the medical observation system according to an embodiment may be omitted. Furthermore, the components of the medical observation system according to an embodiment may be appropriately combined.

Further, in the medical observation system according to an embodiment, the term “unit” described above can be read as “means”, “circuit”, and the like. For example, the control unit can be read as control means or a control circuit.

Further, a program to be executed by the medical observation system according to an embodiment is file data in an installable format or an executable format, and is provided by being recorded in a computer-readable recording medium such as a compact disc read only memory (CD-ROM), a flexible disk (FD), a compact disc-recordable (CD-R) disc, a digital versatile disk (DVD), a universal serial bus (USB) medium, or a flash memory.

Further, the program to be executed by the medical observation system according to an embodiment may be stored in a computer connected to a network such as Internet or may be provided by being downloaded via the network.

Note that although the context between the steps has been described using expressions such as “first”, “then”, and “next” in the flowchart of the present specification, but the order of processing necessary to carry out the present disclosure is not uniquely defined by the expressions. That is, the order of processing in the flowchart of the present specification can be changed as long as it is not inconsistent with the present disclosure.

According to the present disclosure, it is possible to display an image with an appropriate brightness even when the type of the image is switched.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A display device for observing a three-dimensional image or a two-dimensional image through stereoscopic glasses, the display device comprising:
a display panel configured to display a three-dimensional image based on a three-dimensional image signal or a two-dimensional image based on a two-dimensional image signal;
circuitry configured to
determine whether or not an input image signal is the three-dimensional image signal;
change a brightness of an image to be displayed on the display panel to a brightness suitable for the two-dimensional image when it is determined that the input image signal is not the three-dimensional image signal; and
control the display panel to display an image based on the image signal with the changed brightness.

2. The display device according to claim 1, wherein the circuitry further configured to
determine whether or not the brightness of the image to be displayed on the display panel is equal to or lower than a predetermined threshold,
change the brightness of the image to be displayed on the display panel to a brightness suitable for the three-dimensional image when it is determined that the input image signal is the three-dimensional image signal and that the brightness of the image to be displayed on the display panel is equal to or less than the predetermined threshold.

3. The display device according to claim 1, wherein the stereoscopic glasses are passive type glasses.

4. The display device according to claim 1, wherein the circuitry is configured to change the brightness of the image to be displayed on the display panel to a brightness at least twice that for the three-dimensional image when it is determined that the input image signal is not the three-dimensional image signal.

5. A medical observation system comprising:
the display device according to claim 1;
an observation apparatus configured to generate three-dimensional image data by magnifying and capturing an image of a minute structure of an object to be observed; and
a controller configured to performs image processing on the three-dimensional image data and output the processed three-dimensional image data to the display device.

6. A display method executed by a display device including a display panel for observing a three-dimensional image or a two-dimensional image through stereoscopic glasses, the display method comprising:
determining whether or not an input image signal is a three-dimensional image signal;
changing a brightness of an image to be displayed on the display panel to a brightness suitable for the two-dimensional image when it is determined that the input image signal is not the three-dimensional image signal; and
controlling the display panel to display an image based on the image signal with the changed brightness.

7. A non-transitory computer readable recording medium on which an executable program for observing a three-dimensional image or a two-dimensional image through stereoscopic glasses medical image processing, the program instructing a processor of a computer to execute:
determining whether or not an input image signal is a three-dimensional image signal;
changing a brightness of an image to be displayed on the display panel to a brightness suitable for the two-dimensional image when it is determined that the input image signal is not the three-dimensional image signal; and
controlling the display panel to display an image based on the image signal with the changed brightness.
